(19) United States
(12) Patent Application Publication
(10) Pub. No.: US 2008/0311090 A1
(43) Pub. Date: Dec. 18, 2008

(54) Methods for inhibition of proliferative disease, including hepatocellular carcinoma

(75) Inventors: Jer-Yuh Liu, Taichung City (TW); Yi-Hsien Hsieh, Kaohsiung City (TW)

Correspondence Address: WPAT, PC;INTELLECTUAL PROPERTY ATTORNEYS, 2030 MAIN STREET, SUITE 1300, IRVINE, CA, 92614, US

(73) Assignee: CHUNG SHAN MEDICAL UNIVERSITY, Taichung City (TW)

(21) Appl. No.: 11/764,130

(22) Filed: Jun. 15, 2007

(51) Int. Cl. A61K 35/12; A61K 31/7052; A61P 35/00; C12Q 1/68

(52) U.S. Cl. 424/93.21; 435/6; 514/44; 514/789

(57) ABSTRACT

The present invention is related to a method for modulating expression of protein kinase C-alpha associated with proliferative diseases. The invention further relates to a screening method utilizing the association between transcription factors (MZF-1 and Elk-1) and their DNA binding element (PKC-.alpha. promoter) to identify novel anticancer agents.

### FIELD OF THE INVENTION

This invention is related to the field of proliferative disease treatment and diagnosis, particularly human hepatocellular carcinoma and other proliferative diseases that express altered level of protein kinase C-.alpha. This invention further relates to antisense oligonucleotide compounds, particularly the combination of MZF-1 and Elk-1 antisense oligonucleotides, to modulate PKC-.alpha. expression in a tissue of mammal. A screening method disrupting the association between transcription factors and its promoter is also included.

### BACKGROUND OF THE INVENTION

Hepatocellular carcinoma (HCC) is derived from progressive genomic alterations of hepatocellular phenotype. In the early stages, patients with chronic hepatitis and/or cirrhosis exhibit hepatocyte cycling through mitogenic pathways or epigenetic mechanisms. The formation of abnormal dysplastic hepatocytes usually reveal as defective telomere and telomerase, unstable microsatellite, and aberrant structure of genes and chromosomes. Emergence of hepatocellular carcinoma is associated with the accumulation of series disruption of genes involved in various regulatory pathways. To date, molecular marker of hepatocellular carcinoma is not clear yet.

Protein kinase C (PKC) is an important family of signaling molecules modulating cell proliferation, differentiation, transformation, and apoptosis. Ten PKC isoforms are categorized into conventional (cPKCs: alpha, beta I, beta II, and gamma), novel (nPKCs: delta, epsilon, theta, and eta), and atypical (aPKCs: zeta and iota/lambda) subclasses, depending on their requirement for Ca²⁺, phosphatidylserine, and diacylglycerol. Various PKC isoforms differ in their substrate specificity, cellular and subcellular distribution, and tissue specific expression, and it is likely that differential activation of PKC isoforms by second messengers such as diacylglycerol, arachidonic, and phosphoinositides plays a unique role in their regulation and function. Although changes in PKC isoforms are important for the progression of various cancers, the involvement of these enzymes in human HCC remains unclear.

Elk-1 (Ets-like protein-1) is a transcription factor as a member of the ternary complex factor (TCF) subfamily of Ets domain proteins. Elk-1 is involved in regulating cell proliferation, differentiation, and development through mitogen-activated protein kinases (MAPK) pathway. TCFs are able to form a ternary complex with the serum response factor (SRF) and the serum-response element (SRE), and are involved in SRD-driven gene expression. As a subgroup of TCFs, Ets protein family members, Elk-1, Sap1, and Sap2, possess an Ets domain and a winged helix-loop-helix (HLH) DNA binding domain recognizing specific DNA sequences. It has been found that the N-terminal Ets-DNA binding domain of Elk-1 is important for DNA recognition, a B domain containing 20 amino acids mediates protein-protein interaction, and C-terminal of Elk-1 possesses phosphorylation sites for ERK, JNK, and p38 MAPKs. Elk-1 is mostly activated by ERK proteins, and binds to the SRE modulating immediate early gene expression in signaling pathway.

MZF-1 (myeloid zinc finger-1) is a transcription factor of the Kruppel family of zinc finger proteins, which play crucial roles in regulating normal hemopoiesis, originally identified from a cDNA library of chronic myeloid leukemia patient. Transient transfection of MZF-1 in vitro can modulate transcription in hemopoietic and non-hemopoietic derived cells. However, the biological function of MZF-1 is not clear yet.

Recent study has demonstrated a positive correlation between hepatocellular carcinoma and elevated level of PKC-.alpha (Clinca Chimica acta 2007, 382: 54-58). Direct modulators of PKC-.alpha. have yet been examined carefully. The present invention provides methods for monitoring and diagnosing hepatocellular carcinoma via the level of PKC-.alpha. in vitro and in vivo.

Previous discovery has revealed that transcription factors MZF-1 or Elk-1 are associated with PKC-.alpha. promoter with in vitro assay (BBRC 2006, 339: 217-225). The present invention further characterizes the association between MZF-1, Elk-1 and PKC-.alpha. promoter in detail. The association between transcription factor and its promoter provides a new target for screening anticancer drugs which will be discussed as well.

### SUMMARY OF THE INVENTION

The present invention relates to a method for inhibiting proliferative disease with elevated level of PKC-.alpha., especially hepatocellular carcinoma.

This invention further comprises methods of application of PKC.-alpha. modulators, particularly MZF-1 and Elk-1, to inhibit hepatocellular carcinoma.

### BRIEF DESCRIPTION OF THE DRAWINGS

FIG. 1 shows the strong association between PKC-.alpha. and human hepatocellular carcinoma cancerous tissues. Various PKC isoforms are also examined in this figure. Relative mRNA expression levels (PKC isoform/β₂-MG) were expressed as the mean ±SEM. We used an unpaired Student's t-test to analyze the difference in PKC isoform expression levels between HCC and non-cancerous liver tissue. Relative mRNA levels associated with PKC-.alpha., PKC-.iota. and PKC-.delta. isoforms were significantly higher in HCC tissue compared with non-tumorous liver tissue. **: P<0.01; *: P<0.05. White bar: human noncancerous tissue; black bar: human cancerous tissue. Data are shown the mean +/−SE.

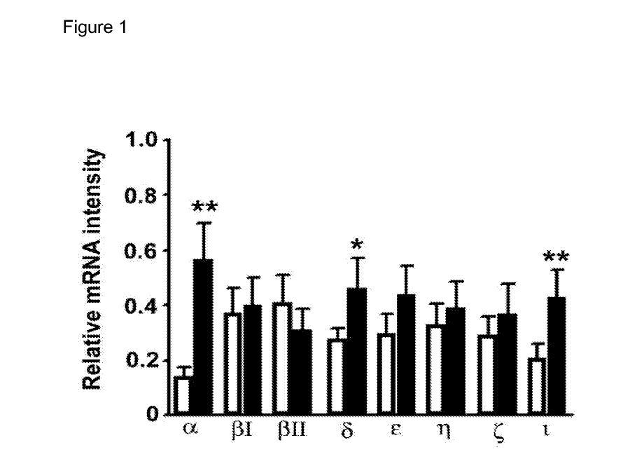

FIG. 2 illustrates the level of PKC-.alpha. mRNA in HA22T/VGH cells treated with antisense or sense oligonucleotides of MZF-1, Elk-1, Egr-1, Oct-1, NF-kB, AP-2. PKC-alpha. mRNA level is reduced in HA22T/VGH cell treated with antisense oligonucleotide of MZF-1 or Elk-1. V: negative control for PCR without RT.

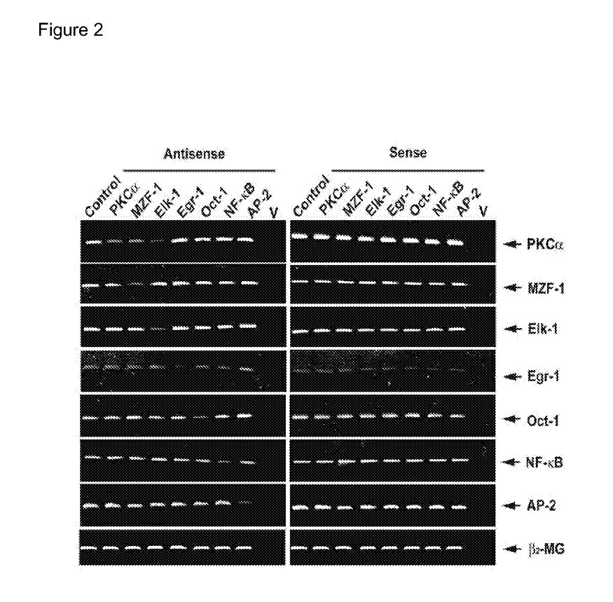

FIG. 3 illustrates reduction of MZF-1 mRNA (A) and protein (B) levels after MZF-1 antisense oligonucleotide treatment of SK-Hep-1 cells. S-MZF-1: sense oligonucleotides of MZF-1; AS-MZF-1: antisense oligonucleotides of MZF-1.

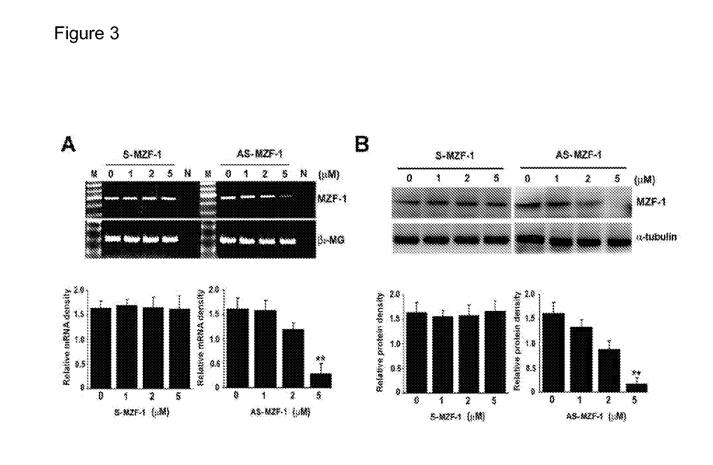

FIG. 4 illustrates specific reduction of PKC-.alpha. mRNA after MZF-1 antisense oligonucleotide treatment of SK-Hep-1 cells. S-MZF-1: sense oligonucleotides of MZF-1; AS-MZF-1: antisense oligonucleotides of MZF-1.

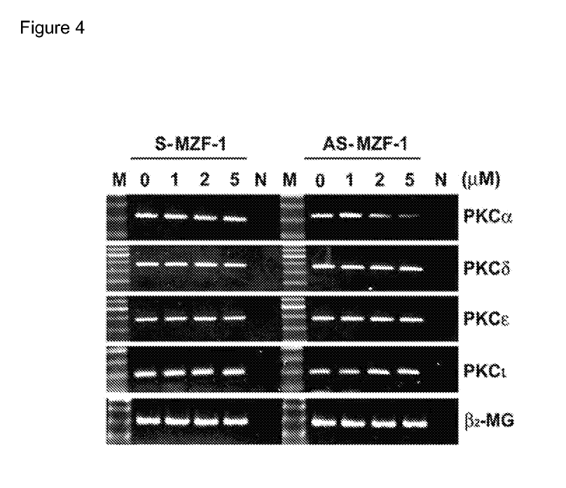

FIG. 5 illustrates effect of antisense ODN MZF-1 on cell growth. The SK-Hep-1 cell was transfected with the indicated dose of antisense or sense ODN MZF-1. Effect of antisense ODN MZF-1 on cell growth. The SK-Hep-1 cells were transfected with either 0, 1, 2 and 5 μM MZF-1 antisense ODN (AS-MZF-1) or 5 μM sense ODN (S-MZF-1). Control, the group was treated with 0 μM MZF-1 antisense ODN. Cell growth was determined 1˜3 days after subculture using the MTT assay as described in Materials and Methods. Absorbance values obtained from untreated cells on day 0 after subculture were taken as 100%. Data are presented as means ±SE of 3 replicates from 2 independent experiments. *P<0.05 versus control; **P<0.01 versus control.

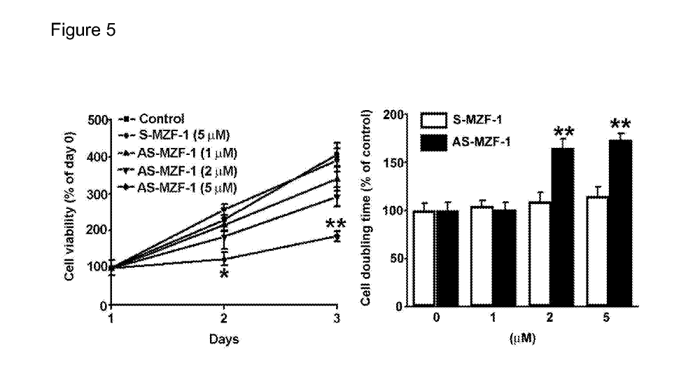

FIG. 6 shows (A) the delayed progression of mice tumor growth curve, (B) and (C) the reduced appearance of mice tumor size treated with antisense oligonucleotide MZF-1-transfected SK-Hep-1 cells. S-MZF-1: sense oligonucleotides of MZF-1; AS-MZF-1: antisense oligonucleotides of MZF-1.

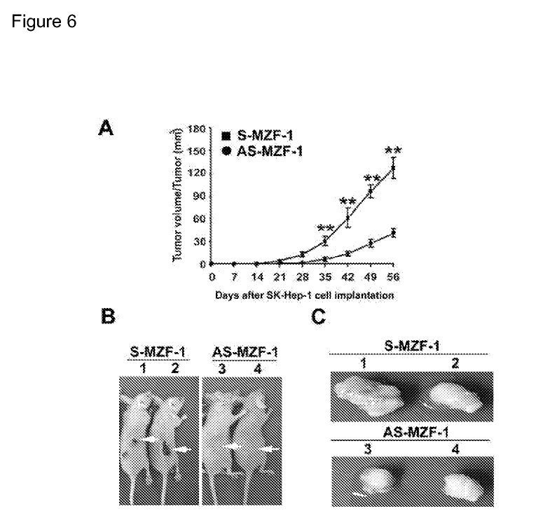

FIG. 7 illustrates reduction of Elk-1 mRNA (A) and protein (B) levels after Elk-1 antisense oligonucleotide treatment of SK-Hep-1 cells. S-Elk-1: sense oligonucleotides of Elk-1; AS-Elk-1, antisense oligonucleotides of Elk-1.

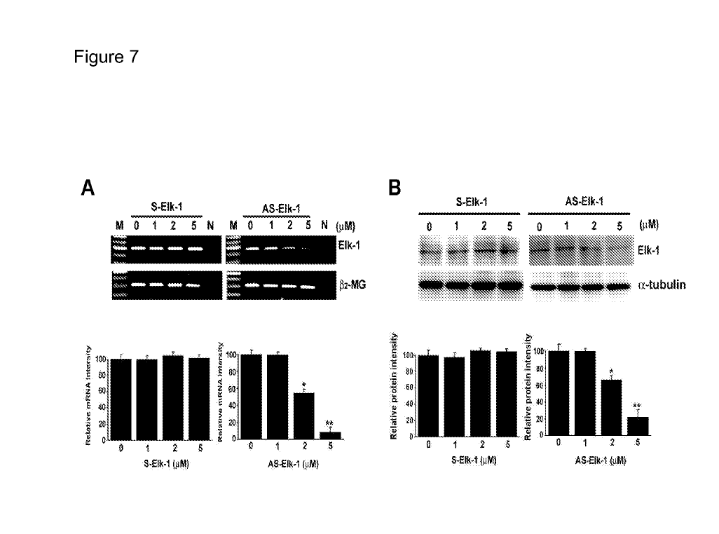

FIG. 8 illustrates specific reduction of PKC-.alpha. mRNA after Elk-1 antisense oligonucleotide treatment of SK-Hep-1 cells. S-Elk-1: sense oligonucleotides of Elk-1; AS-Elk-1, antisense oligonucleotides of Elk-1.

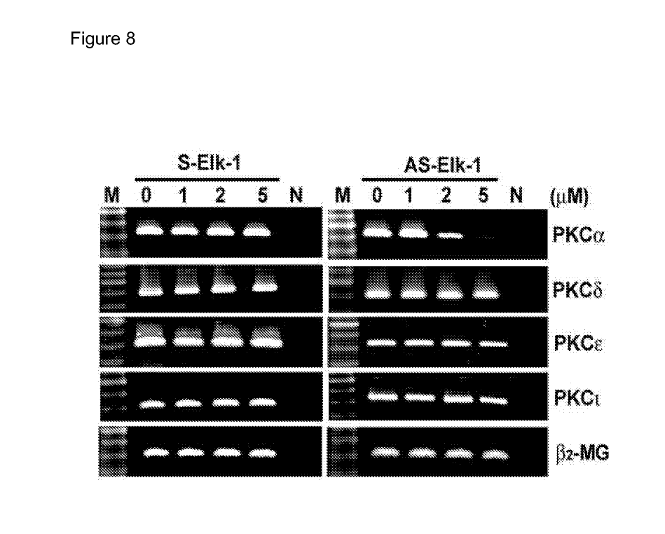

FIG. 9 illustrates effect of antisense ODN Elk-1 on cell growth. The SK-Hep-1 cell was transfected with the indicated dose of antisense or sense ODN Elk-1. Effect of antisense ODN Elk-1 on cell growth. The SK-Hep-1 cells were transfected with either 0, 1, 2 and 5 μM Elk-1 antisense ODN (AS-Elk-1) or 5 μM sense ODN (S-Elk-1). Control, the group was treated with 0 μM Elk-1 antisense ODN. Cell growth was determined 1˜3 days after subculture using the MTT assay as described in Materials and Methods. Absorbance values obtained from untreated cells on day 0 after subculture were taken as 100%. Data are presented as means ±SE of 3 replicates from 2 independent experiments. *P<0.05 versus control; **P<0.01 versus control.

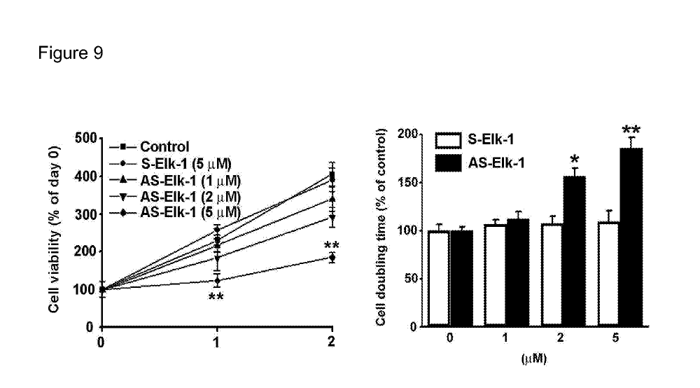

FIG. 10 shows (A) the delayed progression of mice tumor growth curve, (B) and (C) the reduced appearance of mice tumor size treated with antisense oligonucleotide Elk-1-transfected SK-Hep-1 cells. S-Elk-1: sense oligonucleotides of Elk-1; AS-Elk-1, antisense oligonucleotides of Elk-1.

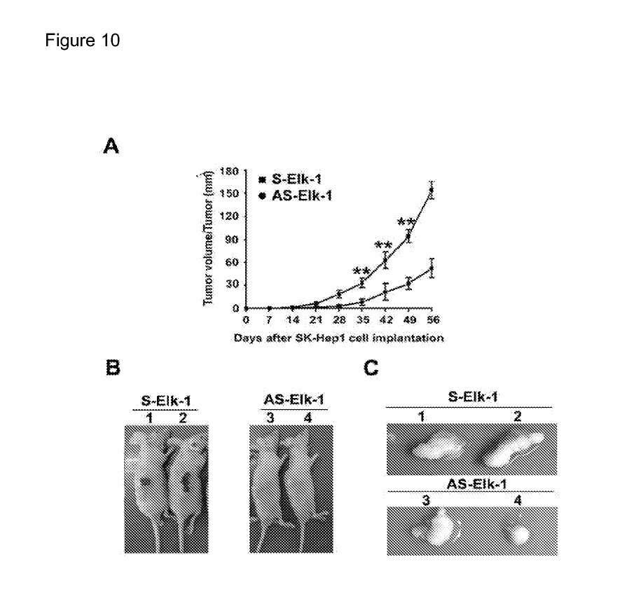

FIG. 11 illustrates antisense Elk-1 and MZF-1 effects on migration and invasion in human HCC cell line HA22T/VGH. The migration (A), and invasion (B) assays were performed on cell cultures treated with antisense Elk-1 (5 μM) (AS-Elk-1) or Elk-1 sense ODN (5 μM) (S-Elk-1), or with antisense MZF-1 (5 μM) (AS-MZF-1) or MZF-1 sense ODN (5 μM) (S-MZF-1) as described in Materials and Methods. Untreated cultures were designated as controls (Control).

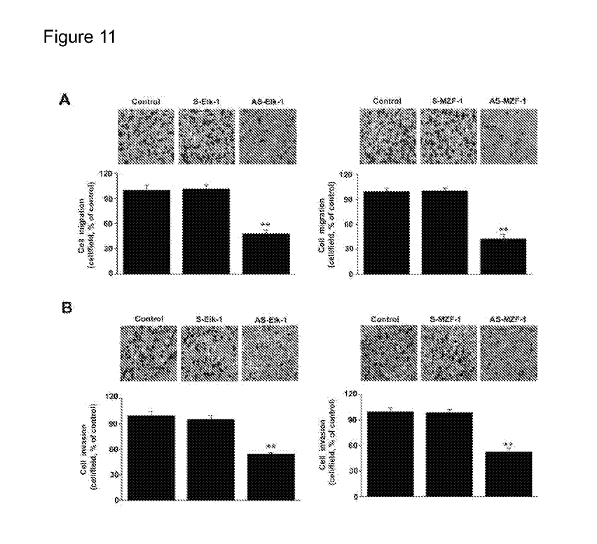

FIG. 12 illustrates PKC-.alpha., MZF-1, and Elk-1 mRNA (A) and protein (B) level in five human liver cancer cell lines, such as poorly-differentiated cells HA22T/VGH, SK-Hep-1, and highly-differentiated cells Huh-7, Hep3B, HepG2. Poorly differentiated hepatocellular carcinoma cells have elevated PKC-.alpha. MZF-1, and Elk-1 than highly differentiated cells.

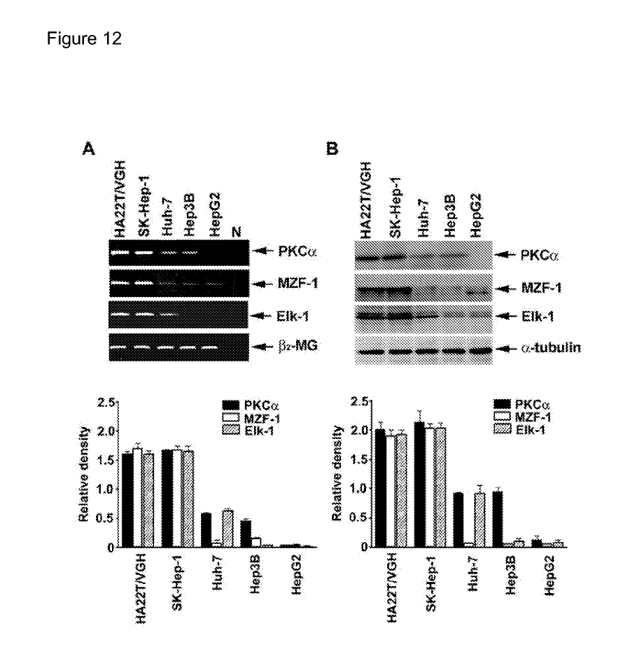

FIG. 13 shows an in vitro system of luciferase driven by PKC-.alpha. promoter (upper panel) and the PKC-.alpha. transcriptional activity activated by MZF-1 or Elk-1 (lower panel). pGL: control vector; IB: immunoblot.

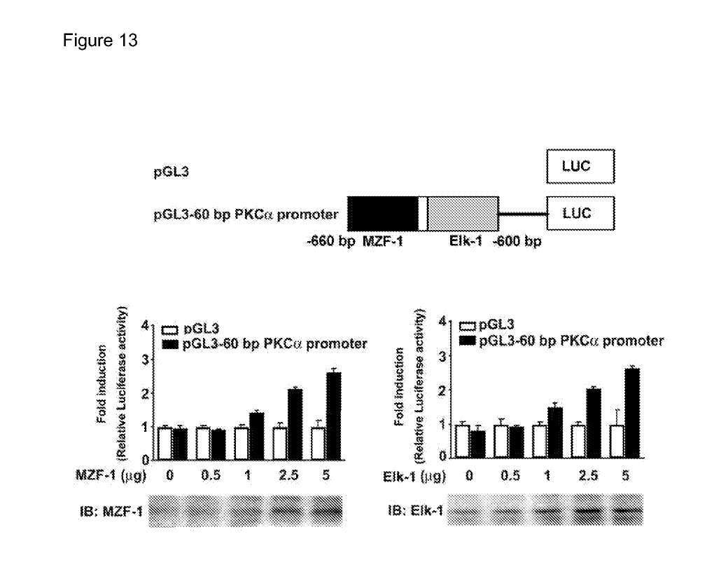

FIG. 14 illustrates the synergistic effect of MZF-1 and Elk-1 of activating luciferase activity driven by PKC-.alpha.-60 bp promoter in various hepatocellular carcinoma cells.

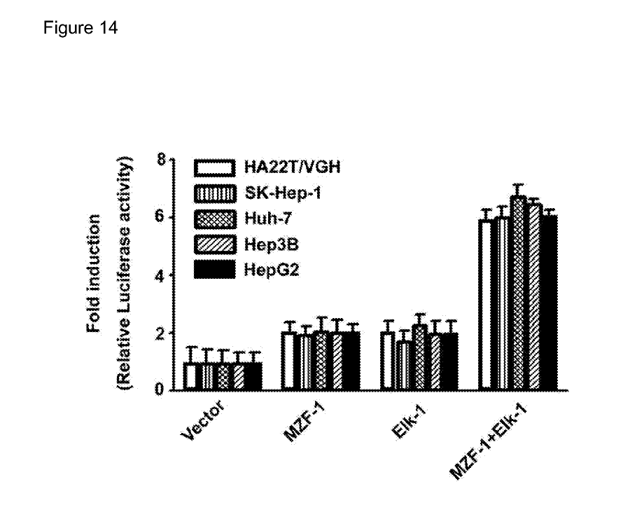

FIG. 15 illustrates the reduction of luciferase activity due to the failure of activating PKC-.alpha. promoter with MZF-1 or Elk-1 lacking DNA binding domain.

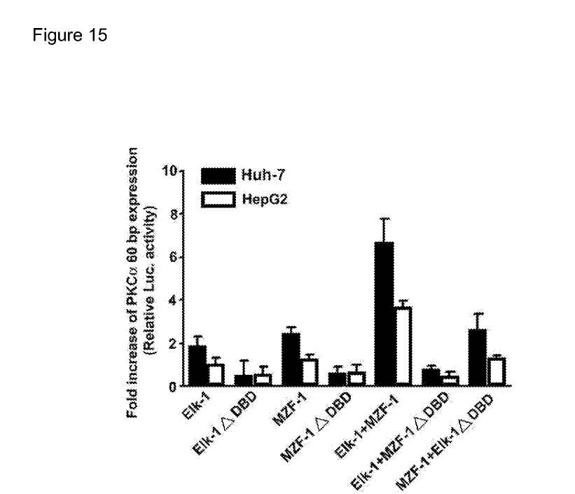

FIG. 16 shows the reduction of luciferase activity due the failure of activating PKC-.alpha. promoter with mutated MZF-1 or Elk-1 binding site on PKC-.alpha. promoter.

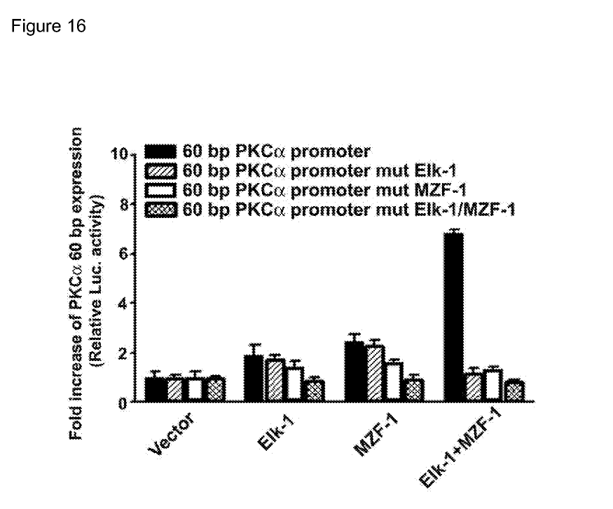

FIG. 17 illustrates identification of a DNA-protein complex in MZF-1/Elk-1 oligonucleotide. (A) MZF-1/Elk-1 oligonucleotides, mut MZF-1/Elk-1 oligonucleotides, MZF-1/mut Elk-1 oligonucleotides or mut MZF-1/mut Elk-1 oligonucleotides sequences. (B) These labeled-oligonucleotides were incubated without (P) or with (N) nuclear extract (10 μg/sample) at room temperature for 15 mins. The reactions were resolved on a 4% nondenaturing polyacrylamide gel. Specific DNA-protein complexes are indicated by arrows. FP, designed as free probe.

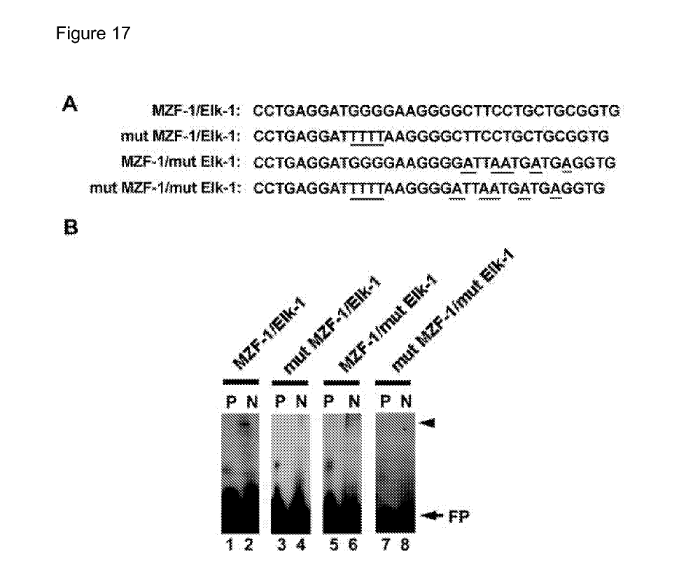

FIG. 18 illustrates association of the MZF-1 and Elk-1 complex with the PKCα promoter. (A) ChIP assay was performed using anti-MZF-1 or anti-Elk-1 antibody on SK-Hep-1 cells. (B) MZF-1 and Elk-1 direct and reverse Re-ChIPs was detected by PCR as described in Materials and methods. (C) ChIP assay was performed on SK-Hep-1 cells transfected with 5 μM sense or antisense of MZF-1, or with 5 μM sense or antisense of Elk-1. PCR was performed on ChIP assay with anti-MZF-1 or anti-Elk-1 antibody. (D). Using reverse Re-ChIPs was detected by PCR as described in Materials and methods. Input represents the purified input-chromatin for parallel PCR reaction. ChIP with nonimmune IgG was used as a control (IP: IgG). The data represent one of three independent experiments with similar results.

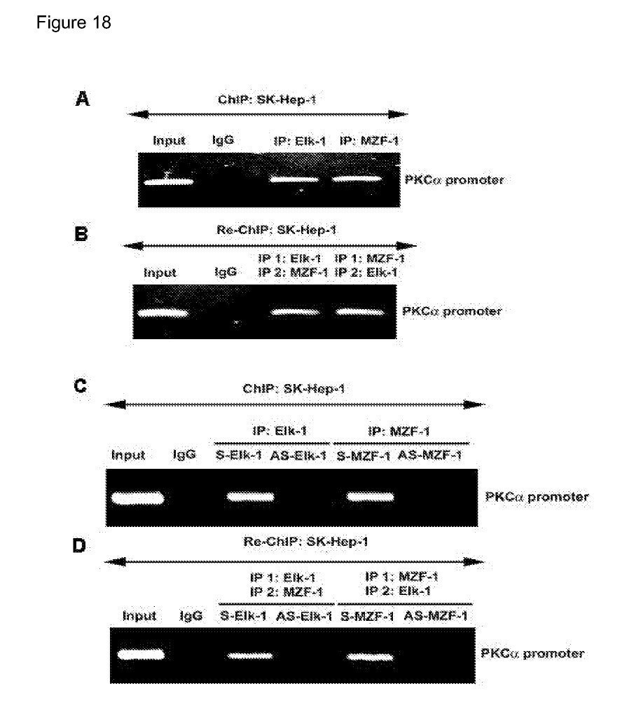

FIG. 19 illustrates reduction of PKC-.alpha. mRNA and protein levels with overexpressed flag-MZF-1 (1-72) (A, B) and c-myc-Elk-1 (86-318) (C, D) treatment in SK-Hep-1 cells.

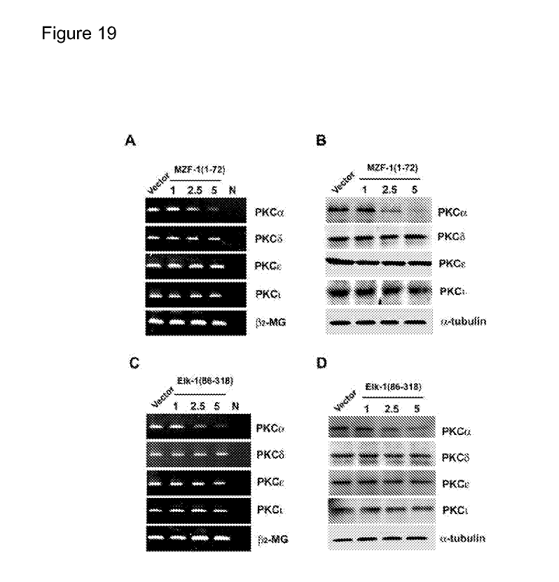

FIG. 20 shows (A) the reduced appearance of mice tumor and (B) the delayed progression of mice tumor growth curve after treated with flag-MZF-1 (1-72)-transfected SK-Hep-1 cells.

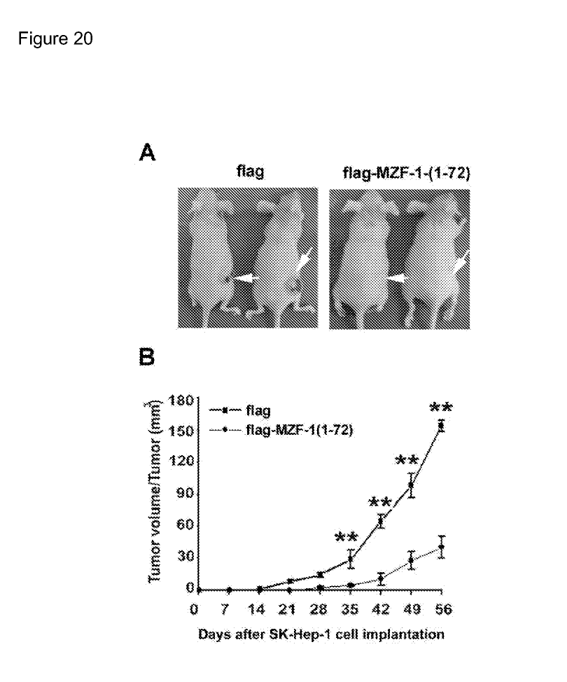

FIG. 21 shows the proposed model for the inhibitory effect of the Elk-1 or MZF-1 proteins on the activity of PKC-.alpha. transcription.

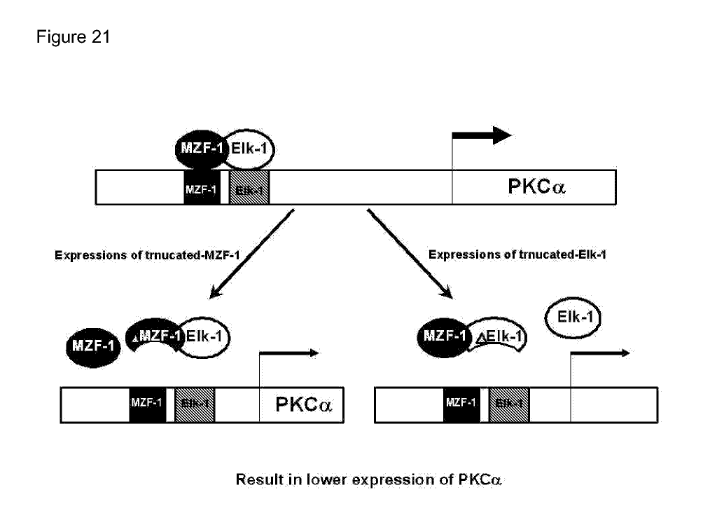

### DETAILED DESCRIPTION OF THE INVENTION

The present invention first reveals that the expression of PKC-.alpha. is associated with the level of transcription factor MZF-1 and Elk-1. Modulation the level of PKC-.alpha. can be achieved by introducing antisense MZF-1, antisense Elk-1, overexpressed polypeptide of MZF-1, overexpressed polypeptide of Elk-1 into a cell.

The present invention further discovers the interaction between MZF-1, Elk-1, and PKC-.alpha. promoter. MZF-1 and Elk-1 form a complex and bind to the promoter of PKC-.alpha. to regulate PKC-.alpha. expression.

The term “MZF-1” used herein indicates a PKC-.alpha. modulator which is a gene or a polypeptide in a full-length form or a truncated form to regulate PKC-.alpha. expression.

The term “Elk-1” used herein also indicates a PKC-.alpha. modulator which is a gene or a polypeptide in a full-length form or a truncated form to regulate PKC-.alpha. expression.

In preferred embodiment, a method of inhibiting the PKC-.alpha. modulators were developed to regulate PKC-.alpha. expression. Modulation of PKC-.alpha. expression may be used as a treatment or diagnosis of proliferative diseases such as cancers in various tissues of ovary, breast, prostate, liver and etc. In preferred embodiment, the modulators of PKC-.alpha. are antisense oligonucleotides specifically hybridizable with nucleic acid(s) encoding MZF-1 or Elk-1. The present invention also includes overexpression of PKC-.alpha. modulators specifically interacting with PKC-.alpha. promoter to control its expression.

Oligonucleotides have been employed as therapeutic agents for the treatment of diseased states in mammals. Antisense, triplex and other oligonucleotide compositions are capable of modulating expression of genes caused by viral or fungal infection and metabolic disorder.

Antisense oligonucleotides have been developed as therapeutic drugs of mammals for various diseases. Targets of the antisense oligonucleotides may include viral and cellular gene products. As used herein, the cellular gene target comprises MZF-1, Elk-1, and PKC-.alpha. in a form of DNA (untranscribed region and transcribed region), RNA (pre-mRNA and mRNA), or protein (whole polypeptide and truncated polypeptide).

In present invention, “modulation” means either an increase (stimulation) or a decrease (inhibition) in the expression of a gene, particularly PKC-.alpha. In preferred embodiments, inhibition is the preferred form of modulation of gene expression and mRNA is a preferred target via antisense oligonucleotides, particularly MZF-1, Elk-1, or both. This modulation of gene expression can also be achieved by overexpressing truncated polypeptides of PKC-.alpha.'s modulators, particularly MZF-1, Elk-1, or both. The modulation of PKC-.alpha. expression can be measured with routine techniques in any laboratory, such as northern blot assay or reverse transcriptase PCR for determining mRNA expression, or western blot, ELISA or immunoprecipitation assay for determining protein expression.

The hybridizable regions of antisense oligonucleotides may comprise various parts of messenger RNA from specific genes, such as 5′-untranslated region, coding sequence region (intron and exon), and 3′-untranslated region. The antisense oligonucleotides may specifically hybridize with any part of the messenger RNA from specific gene in order to inhibit a downstream gene expression. The antisense oligonucleotides may recognize a reserved region of target gene among a gene family, or a diversified region among a gene family depending on the desired effectiveness of one administration. As used herein, the specific genes are modulators of PKC-.alpha., particularly MZF-1 and Elk-1, and the downstream gene is PKC-.alpha.

In the present invention, hybridization means hydrogen bonding formation between complementary bases followed the rule of well-known Watson-Crick base pairing. For example, guanine (G) and cytosine (C) are complementary bases forming three hydrogen bonds between them, and adenine (A) and thymine (T) are examples of complementary bases forming two hydrogen bonds between them.

The present invention is suitable for diagnosing proliferative disease in tissue or other samples from patients suspected of having a cancer in liver, lung, breast, prostate and etc. Several assays may be formulated employing the present invention, usually comprising a tissue sample with an oligonucleotide of the invention under conditions for quantification of such inhibition.

The oligonucleotides of present invention may be used for research purposes, such as investigating the function of a specific gene product in a signaling pathway with specific oligonucleotides. A specific hybridization process with oligonucleotides may be applied for assays, purifications, cellular product preparations and etc.

The oligonucleotide compounds in the present invention comprise from about 5 to about 50 nucleobases. Antisense oligonucleotides comprising from about 8 to about 30 nucleobases are preferred embodiments. As is known in the art, an oligonucleotide comprises sugar backbone, base, and phosphodiester bond building by a chain linkage of nucleosides. An open linear structure is preferred, as well as a 5′ to 3′ phosphdiester linkage of a oligonucleotide.

Oligonucleotides in present invention may also include modified oligonucleotides. Modified oligonucleotides are oligonucleotides contain at least one region wherein the oligonucleotide is modified so as to confer upon the oligonucleotide increased resistance to nuclease degradation, increased cellular uptake, and/or increased binding affinity for the target nucleic acid. Modified oligonucleotides may also be used as a substrate for enzymes capable of cleaving RNA: DNA or RNA: RNA hybrids. For example, activation of RNase H, a cellular endonuclease for cleaving the RNA strand of an RNA: DNA duplex, is able to enhance the inhibition of target gene expression via antisense oligonucleotides. Examples of modified oligonucleotides include but not limit to oligonucleotides modified with nucleotide (substitution, insertion, deletion and etc), sugar backbone, base, phosphate group, and other linkages directly or indirectly connected to oligonucleotides.

The oligonucleotides used in present invention may be routinely made through the well-known technique of solid phase synthesis or similar techniques to synthesize oligonucleotides or fluorescent-, biotin-, and other conjugates-labeled oligonucleotides. Bioequivalent compounds such as acceptable salt may also be included in the present invention.

In present invention, polypeptides of MZF-1, Elk-1, or both for administrating into a diseased animal may be generated from various vectors containing promoters with different transcriptional or translational strength. Various promoters may exhibit the ability synthesizing different amounts of polypeptide, such as the amount less than endogenous polypeptide, equivalent to endogenous polypeptide, more than endogenous polypeptide, and etc. The amount and effectiveness of various polypeptides can be measured by routine laboratory techniques such as ELISA, western blot analysis, immunoprecipitation and etc. The preferred example of the amount of polypeptide expressing from various promoters is the amount of polypeptide more than endogenous ones.

In present invention, overexpressed polypeptide may be modified from full-length polypeptide, such as substitution, deletion, insertion and etc. Other commercially available protein tags may also be included in various parts of the overexpressed polypeptide such as N-terminal end, internal sequence, C-terminal end, and etc. One preferred example is flag tagged MZF-1 on the N-terminal region. Another example is the myc tagged Elk-1 on the N-terminal region.

Depending on the type of a disease, the compositions of the present invention may vary upon a requirement of local or systemic treatment, or upon an area to be treated. Administration of the present invention may be topical (including ophthalmic, vaginal, rectal, intranasal, epidermal, and transdermal), oral or parenteral (including intravenous drip, subcutaneous, intraperitoneal or intramuscular injection, pulmonary administration and etc).

Compositions for oral administration comprises powders or granules, suspensions or solutions in aqueous or non-aqueous media, capsules, tablets, and other desirable thickeners, flavoring agents, diluents, emulsifiers, dispersing aids or binders. Compositions for parenteral administration may comprise sterile aqueous solutions containing buffers, diluents and other suitable additives. Traditional therapeutic agents may be combined with oligonucleotides or polypeptides for an effective treatment of an animal.

Dosing is dependent on several features of a diseased animal, such as the severity and responsiveness of the disease, the duration of treatment, and other features which are routinely considered during treatment of an animal. Optimum dosages may vary depending on the potency of individual oligonucleotides or polypeptides. In general, dosage is from 0.01 μg to 100 g per kg of body weight, and may be given once or more daily, weekly, monthly or yearly, or even once every 2 to 20 years. Such dosages are an effective amount effective to modulate PKC-.alpha. expression. The maintenance therapy may be applied to prevent the recurrence of a disease, wherein the oligonucleotides or polypeptides are administered in maintenance doses, ranging from 0.01 μg to 100 g per kg of body weight, once or more daily, up to once every 20 years.

The meaning of therapeutically effective amount is the amount of the compound applied to a patient to show therapeutic effects. This amount is variable among the kind of animal, the type of disease, the age, weight, or other related factors of an animal, and other features which are regularly taken into consideration during drug development. The meaning of therapeutic effect is a measurement of the effect after treating a diseased animal with the compound, such as measuring the rate of tumor growth curve or the size and volume of the tumor, or assessing the production of compound as PKC-.alpha. which is an molecular indicator of the tumor.

The present invention further characterizes the interaction between MZF-1, Elk-1, and PKC-.alpha. promoter. Complex of MZF-1 and Elk-1 may bind to the promoter of PKC-.alpha. in order to regulate PKC-.alpha. expression and modulate proliferative disease progression.

Polypeptides of MZF-1 or Elk-1 in the present invention may function as a monomer, homodimer or heterodimer to modulate target gene expression such as PKC-.alpha. In preferred embodiments, heterodimer of MZF-1 and Elk-1 is able to associate with the promoter region of PKC-.alpha. in order to activate PKC-.alpha. expression. Overexpressed MZF-1 or Elk-1 polypeptides may disrupt this homeostasis of protein-DNA interaction, which may lead to abnormal expression of PKC-.alpha. Modified MZF-1 and Elk-1 polypeptides lacking DNA binding domain as well as the protein-protein interaction domain may also cause abnormal expression of PKC-.alpha. expression. Another example is that the MZF-1 or Elk-1 binding site on PKC-.alpha. promoter may be modified to block the association between polypeptide of MZF-1 and PKC-.alpha. promoter or polypeptide of Elk-1 and PKC-.alpha. promoter. Essential elements of PKC-.alpha. promoter for MZF-1 and/or Elk-1 binding may be replaced, deleted, or modified in order to modulate PKC-.alpha. expression.

The present invention further comprises a screening method to identify compounds, ligands, polypeptides or other agents for inhibiting the association between polypeptide MZF-1, polypeptide Elk-1, and PKC-.alpha. promoter.

In preferred embodiment, the screening method is utilized to identify novel anticancer agents which target the interaction between transcription factors such as MZF-1 and Elk-1 and PKC-.alpha. promoter. Factors associated with MZF-1 or Elk-1 are potential anticancer agents modulating PKC-.alpha. expression. A variety of protocols and techniques may be utilized to screen agents which inhibit or interfere the association between MZF-1 and Elk-1, such as biochemical assay, biophysical assay, yeast two-hybrid assay, luciferase complementation. Biochemical assays for identifying these factors include co-immunoprecipitation, GST-pull down, ELISA, tandem affinity purification and other commonly used assays. Biophysical assays for identifying these factors include fluorescent resonance energy transfer assay, surface plasmon resonance using Biacore, isothermal calorimetric analysis, atomic force microscopy, quartz crystal microbalance biosensor and other commonly used assays.

In preferred embodiment, a screening method comprises an agent to be tested, polypeptide MZF-1, polypeptide Elk-1, and a reporter gene driven by a PKC-.alpha. promoter in a cell. The PKC-.alpha. promoter comprises the essential elements for the binding of transcription factors such as MZF-1 and Elk-1. The effectiveness of the testing agent can be measured by the expression level of reporter gene driven by PKC-.alpha. promoter.

The reporter gene utilized in the screening method comprises luciferase, green fluorescent protein, beta-galactosidase, beta-glucuronidase, beta-lacatamase, chloramphenical acetyltransferase and other commonly used reporter genes. The more suitable reporter gene comprises luciferase, green fluorescent protein, and beta-galactosidase. The most suitable reporter gene is luciferase.

The PKC-.alpha. promoter utilized in the screening method comprises a 60 bp fragment from −600 bp to −660 bp of PKC-.alpha gene. The 60 bp PKC-.alpha. promoter fragment comprises two parts, such as MZF-1 binding site and Elk-1 binding site. Mutations may be introduced into either or both parts of the fragment to disrupt the binding of transcription factors, such as MZF-1 or Elk-1. The disruption of the transcription factors binding may lead to the abnormal expression of a reporter gene driven by the PKC-.alpha. promoter.

The agents identified from this novel anticancer screening method not only provide a potential anticancer drug with reduced effective dose and toxicity but also improve the absolute anticancer effect through a variety of mechanisms.

The following examples illustrate the present inventions and are not limited to the same.

### EXAMPLES

### Example 1

### Plasmid Construction

The expression vectors described below were driven by the cytomegalovirus (CMV) promoter-basic contained in the pcDNA3 vector (Invitrogen, Carlsbad, Calif.). The entire open reading frames of human MZF-1 and Elk-1 genes were obtained from the SK-Hep-1 cells by RT-PCR. pcDNA-Elk-1 (GenBank Accession No. NM005229, 234-1520) and pcDNA-MZF-1 (GenBank Accession No. M58297, 1091-2548) were amplified by PCR using the primer pairs 5′-TTATAAGCTTATGGACC CATCTGTGACGCT-3′ as SEQ ID No:1 encoding nucleotides 234-253 followed by a HindIII site and 5′-TTATGGATCCTCATGGCTTCTGGGGCCCT-3′ as SEQ ID No:2 encoding nucleotides 1520-1502 followed by a stop codon (TGA) and a BamHI site, and 5′-TTATAAGCTTTGTCATGAATGAATGGT-3′ as SEQ ID No:3 encoding nucleotides 1075-1091 followed by a HindIII site and 5′-TTATCTCGAGCTACTCGGCGCTGTGGA-3′ as SEQ ID No:4 encoding nucleotides 2532-2548 followed by a stop codon (TGA) and a XhoI site, respectively. PCR amplifications were performed with Super-Therm DNA polymerase (Promega) employed 35 cycles with steps at 94 degree C. for 30 seconds, 52.degree C. for 30 seconds, and 72.degree C. for 2.5 minutes. The PCR products were isolated and cloned into the PGEM-T Easy vector (Promega). pcDNA-Elk-1 was constructed by digesting pGEM-T-Elk-1 with HindIII and BamHI, and the 1286 bp fragment was isolated and cloned into the HindIII-BamHI sites of the pcDNA3 vector. pcDNA-MZF-1 was constructed by digesting PGEM-TMZF-1 with HindIII and XhoI, and the 1457 bp fragment was isolated and cloned into the HindIII-XhoI sites of the pcDNA3 vector. Sequence fidelity of both Elk-1 and MZF-1 was confirmed using DNA sequence analysis.

Vector containing flag-MZF-1 (1-72) was constructed by expressing MZF-1 (1-72aa; contains 1-216 bp) in a PFLAG-CMV vector. Fragment of MZF-1 (1-72) as SEQ ID No: 31 (MZF-1ΔDBD) was amplified by RT-PCR from RNA obtained from SK-Hep-1 cells. Primer pairs are 5′-TGAATTCATGAATGGTCCCCTTGTG-3′ as SEQ ID No:27 and 5′-TTCTAGAGCAGGGATCCTCGTCCGT-3′ as SEQ ID No:28 with a EcoRI and XbaI site, respectively. The PCR products were isolated and cloned into the PGEM-T Easy vector (Promega). pFlag-MZF-1 (1-72) was constructed by digesting pGEM-T-MZF-1 (1-72aa) with EcoRI and XbaI, and the 216 bp fragment was isolated and cloned into the EcoRI-XbaI sites of the PFLAG-CMV vector.

Vector containing myc-Elk-1 (86-318) was constructed by expressing Elk-1 (86-318aa; contains 258-954 bp) in a pcDNA-myc vector. Fragment of Elk-1 (86-318) as SEQ ID No: 32 (Elk-1ΔDBD) was amplified by RT-PCR from RNA obtained from SK-Hep-1 cells. Primer pairs are 5′-CAGCAGCACCATGTCCCACA-3′ as SEQ ID No: 29 and 5′-TGTGGGACATGGAGCTGCTG-3′ as SEQ ID No:30 with a BamHI and HindIII site, respectively. The PCR products were isolated and cloned into the PGEM-T Easy vector (Promega). pMYC-Elk-1 (86-318) was constructed by digesting pGEM-T-Elk-1 (86-318aa) with BamHI and HindIII, and the 696 bp fragment was isolated and cloned into the EcoRI-XbaI sites of the pcDNA-myc vector.

### Example 2

### Cell Culture and Growth Conditions

HA22T/VGH (BCRC No. 60168), PLC/PRF/5 (BCRC No. 60223), Hep3B (BCRC No. 60434), and HepG2 (BCRC No. 60025) were purchased from the Bioresources Collection and Research Center, Food Industry Research and Development Institute (Hsinchu, Taiwan) and SK-Hep-1 was from the American Type Culture Collection (Rockville, Md.). The HA22T/VGH and SK-Hep-1 lines are poorly differentiated hepatocellular carcinoma (HCC) cells, whereas PLC/PRF/5, Hep3B, and HepG2 are well differentiated HCC cells. These cell lines were cultured with DMEM (Gibco-BRL) supplemented with 100 μM non-essential amino acid, 2 mM glutamate, 10% fetal bovine serum (FBS), 100 U/ml penicillin G, and 100 μg/ml streptomycin (Sigma Chemicals, St. Louis, Mo.) in a humidified atmosphere containing 5% CO₂ at 37.degree. C.

### Example 3

### Transfection of Sense and Antisense Oligonucleotides into Cells

Transfections were performed using lipofectin. Cells seeded at 60-mm dish were cultured for 24 hours in DMEM supplemented with 10% FBS, rinsed with serum-free MEM and 1 ml MEM containing 15 μg/ml lipofectin, and 2 or 4 μg pcDNA-Elk-1 or/and pcDNA-MZF-1. The cells were incubated at 37.degree. C. for 6 hours before adding 1 ml MEM supplemented with 20% FBS to the medium. After incubation for 18 hours, the medium was replaced with fresh 10% FBS-DMEM. Cells were lysed 48 hours after transfection with guanidinium thiocyanate buffer for RT-PCR assay and western blot analysis.

### Example 4

### RNA Isolation for Gene Expression Analysis

Total RNA was isolated from cells by the guanidinium thiocyanate-phenol method (Chomczynski et al., 1987, Anal. Biochem., 162, 156-159). The HCC cell lines were homogenized (4M guanidinium thiocyanate, 25 mM sodium citrate, 0.5% (w/v) sodium lauryl sarcosinate, and 0.1 M (-mercaptoethanol) in a polypropylene tube, and then total RNA was isolated using a standard method. Phenol-chloroform reagent was added to the samples, and centrifuged the tube at 12,000 g for 30 min at 4.degree. C., RNA was precipitated from the aqueous phase using isopropanol, and then the resultant pellet was washed twice with 70% ethanol. The RNA content of the resuspended pellet was quantified and checked for purity and condition by spectrophotometry at a wavelength of 260 nm. The extract integrity was assessed by 1.5% agarose gel electrophoresis and RNA was visualized by ethidium bromide staining.

### Example 5

### Reverse Transcriptase

### Polymerase Chain Reaction for Gene Expression Analysis

RT-PCR assay was performed according to De Petro et al. (1998, Cancer Res., 58, 2234-2239) with slight modifications. An aliquot of total RNA (0.5 (g) was reverse transcribed using 0.5 (M oligo d (T) primers in a reaction solution (50 (I) containing 75 mM KCl, 50 mM Tris-HCl (pH 8.3), 3 mM MgCl2, 10 mM DTT, 10 U RNase inhibitor (Promega, Madison, Wis.), 0.8 mM total dNTPs, and 200 U of Moloney murine leukemia virus reverse transcriptase (Promega). The sample was incubated at 42 degree C. for 1 hour and at 99 degree C. for 5 minutes before chilling on ice for 10 minutes. The RT product (2 (I) was diluted with the PCR buffer (50 mM KCl, 10 mM Tris-HCl, and 2 mM MgCl2) to a final volume of 50 (I, containing 0.5 (M dNTPs (final concentration, 0.8 mM) and 0.5 U of Super-Therm Taq DNA polymerase (Southern Cross Biotechnology, Cape Town, South Africa). PCR was performed on a GeneAmp PCR system 2400 (Applied Biosystems, Foster City, Calif.). For each experiment, up to 33 cycles were performed to avoid reaching the PCR plateau values. The PCR products were analyzed by 1.2% agarose gel electrophoresis and direct visualization after SYBR Green I (Cambrex Bio Science Rockland, Rockland, Me.) staining. The agarose gels were scanned and analyzed using the Kodak Scientific 1D Imaging System (Eastman Kodak Company, New Haven, Conn.). The accuracy of the amplification reaction for each set of primers was determined by amplifying several dilutions of the same cDNA with the same cycling profiles and amplifying the same cDNA dilution with different cycling profiles. The specificity of the cDNA was also checked using DNA sequence analysis.

### Example 6

### Immunoblot Assay for PKC Expression

Cell extracts were separated on 10% (w/v) SDS-PAGE gels electrophoretically transferred onto a PVDF membrane (Millipore, Belford, Mass.). The membrane was blocked with 5% (w/v) non-fat dried milk in TBST buffer (20 mM Tris-HCl (pH 7.4), 150 mM NaCl, and 0.1% (v/v) Tween 20) and then incubated with the specific anti-PKC isoform (1:1000) or alpha-tubulin (1:3000) antibody at 4 degree C. overnight. After washing with TBST, the blots were then incubated with HRP-conjugated anti-mouse antibody (1:3000) at room temperature for 1 hour and then washed with TBST before visualized using chemiluminescence (Amersham-Pharmacia Biotech, Piscataway, N.J.). PKC-.alpha. appears as a single band with a molecular weight of 80 kD.

### Example 7

### Antisense Knockdown Assay

The antisense knockdown assay was performed according to Shen et al. (1999, Mol. Pharmacol., 55, 396-402) and the following antisense and sense (as a control) sequences were used: PKC-.alpha. (antisense 5′-GTTCTCGCTGGTGAGTTTCA-3′ as SEQ ID No:5, sense 5′-GGTTTTACCATCGGTTCTGG-3′) as SEQ ID No:6; PKC-.iota. (antisense 5′-TGTGGGACATGGAGCTGCTG-3′ as SEQ ID No:7, sense 5′-CAGCAGCACCATGTCCCACA-3′ as SEQ ID No:8); Elk-1 (antisense 5′-CAGCGTCACAGATGGGTCCAT-3′ as SEQ ID No:9, sense 5′-ATGGACCCATCTGTGACGCTG-3′ as SEQ ID No:10); MZF-1 (antisense 5′-TACACAAGGGGACCATTCATTC-3′ as SEQ ID No:11, sense 5′-GAATGAATGGTCCCCTTGTGTA-3′ as SEQ ID No:12). Egr-1 (antisense 5′-GGGGTAGTTGTCCAT-3′ as SEQ ID No:13, sense 5′-ATGGACAACTACCCC-3′ as SEQ ID No:14); Oct-1 (antisense 5′-GGATTGTTCATTCTTGAGTC-3′ as SEQ ID No:15, sense 5′-GACTCAAGAATGAACAATCC-3′ as SEQ ID No:16); NF-kB (antisense 5′-GGATCATCTTCTGCCATTCTG-3′ as SEQ ID No:17, sense 5′-CAGAATGGCAGAAGATGATCC-3′ as SEQ ID No:18); AP-2 (antisense 5′-GTCAATTTCCAAAGCATTT-3′ as SEQ ID No:19, sense 5′-AAATGCTTTGGAAATTGAC-3′ as SEQ ID No:20). The ODN sequence of PKC-.iota. is in the region 251-270 of human PKC-.iota. mRNA (GenBank Accession No. NM_{—}002740), the MZF-1 ODN sequence is in the region 1089-1110 of human MZF-1 mRNA (GenBank Accession No. M58297), the Oct-1 ODN sequence is in the region 82-101 of the human Oct-1 mRNA (GenBank Accession No. X13403) and the AP-2 ODN sequence is in the region 61-79 of human AP-2 mRNA (GenBank Accession No. X52611). They were formed for targeting the AUG region and had no more than four contiguous intrastrand base pairs or four contiguous G:C pairs. Cells were plated at 70% density 24 hours before antisense ODN treatment. The cells were washed in triplicate with serum-free DMEM and incubated with antisense ODN (0, 0.5, 1.0, 2.0, or 5.0 μM) in serum-free DMEM containing 10 (g/ml lipofectin (Life Technologies, Grand Island, N.Y.) at 37.degree. C. The medium was changed to 10% FBS DMEM 6 hours later before culturing at 37 degree C. for 24 or 48 hours.

### Example 8

### In Vitro Tumorgenesis Assay is Used for Evaluating the Effect of Antisense MZF-1 or ELK-1 in Mice

BALB/cAnN-Foxn mice of four- to six-week-old were used in the experiment. Mice were maintained under sterile condition and fed with sterile water and food. SK-Hep-1 cells were transfected with 5 μM antisense or sense MZF-1, treated with trypsin for 5 minutes, spun at 1000 rpm for 5 minutes, and harvested at a density of 1×10⁷ cells/ml. One hundred microliter of sense or antisense MZF-1 transfected SK-Hep-1 cells was injected subcutaneously into nude mice. Tumor size was measures as follow. Tumor volume=0.5236×L1 (L2)². L1: length of tumor. L2: width of tumor. Tumor suppression rate=(tumor volume_{S-ODN}−tumor volume_{AS-ODN}/tumor volume_{S-ODN})×100%.

### Example 9

### Cell Proliferation Assay is Used for Evaluating the Effect of PKC-.Alpha. Expression in HCC Cells

Cell proliferation was determined by the yellow tetrazolium MTT assay (Sobottka et al., 1992, Cancer Chemother. Pharmacol. 30, 385-393). The cells were seeded in 24-well plates at a density of 1×10⁴ cells/well and cultured in DMEM containing 10% serum overnight. These cells were treated with and without specific antisense ODNs and incubated for 24 or 48 hours. After incubation, the medium was replaced with fresh medium and the cells were incubated with 5 mg/ml MTT for 4 hours before dissolving in 1 ml isopropanol for 10 minutes. The optical density at 570 nm was then measured using a spectrophotometer. Cells at the log phase were used to calculate the doubling time according to the equation doubling time (h)=[log 2×(24×No. of days)]/[log Density_{final}−log Densityᵢₙᵢₜᵢₐₗ].

### Example 10

### Cell Migration and Invasion Assay are Used for Evaluating the Effect of PKC-.Alpha. Expression in HCC Cells

Cells were treated with indicated sense or antisense ODN (5 (M), detached by trypsinization 48 hours later, and then washed in triplicate in serum-free DMEM. For the migration assay (Saurin et al., Cancer Res., 2002, 4829-4835), the cells were plated at 2×10⁵ cells/well in serum-free DMEM in the upper chamber of a 48-well Boyden chamber (Neuro Probe, Gaithersburg, Md.), which was plated with the 8-(m pore size polycarbonate membrane filters (Neuro Probe) for 2 hours before the cells were added. The cells were then incubated in a humidified 5% CO2 atmosphere at 37.degree. C. for 6 hours in HA22T/VGH cells, for 12 hours in SK-Hep-1 cells, and for 24 hours in other cell lines. The invasion assay (Saurin et al., 2002, Cancer Res., 62, 4829-4835) was performed in the same manner as the migration assay, except that the filter was precoated with 10 (g/ml Matrigel (Collaborative Biomedical Products, Bedford, Mass.) and the cells were incubated at the same conditions for 8 hours in HA22T/VGH cells, for 24 h in SK-Hep-1 cells, and for 24 hours on other cell lines. After incubation, the cells were fixed with methanol and stained with 0.05% Giemsa. Those on the upper surface of the filter were removed with a cotton swab. The filters were then rinsed in distilled water until no additional stain leached. The cells were then air-dried for 20 minutes. The number of cells was counted within a field at 200× under a light microscope. For each membrane, a total of 4 fields were selected at random and the numbers were averaged.

### Example 11

To determine whether Elk-1 and MZF-1 were involved in transcriptional regulation of the PKCα, luciferase reporter constructs bearing the PKCα promoterwere transfected into liver cancer cell lines along with Elk-1, MZF-1, both of Elk-1 and MZF-1, MZF-1 or Elk-1 lacking DNA binding domain, Elk-1 and MZF-1 lacking DNA binding domain, or MZF-1 and Elk-1 lacking DNA binding domain. Using β-galactosidase to normalize for transfection efficiencies, we analyzed the luciferase activity driven by the PKCα promoter.

The experiments were performed using HA22T/VGH, SK-Hep-1, Huh-7, Hep3B., and HepG2 cell lines. The cells were grown in 24-well plates. At 70% confluence, the cultures were transfected for 6 h with 0.5 μg of the reporter plasmids, pGL3-60 bp PKC.alpha. promoters (60 bp PKC.alpha. promoter, sequence 5′-ctcgagcacc gggggtcctg aggatgggga aggggcttcc tgctgcggtg ctgaggaagc-3′ as SEQ ID No. 33 containing a putative binding site for MZF-1/Elk-1; 60 bp PKC.alpha. promoter mutMZF-1, sequence 5′-ctcgagcacc gggggtcctg aggattttga aggggcttcc tgctgcggtg ctgaggaagc-3′ as SEQ ID No. 34 containing a putative binding site for mut MZF-1/Elk-1; 60 bp PKC.alpha. promoter mutElk-1, sequence 5′-ctcgagcacc gggggtcctg aggatgggga aggggcttaa ggctgcggtg ctgaggaagc-3′ as SEQ ID No. 35 containing a putative binding site for MZF-1/mut Elk-1; 60 bp PKC.alpha. promoter mutMZF-1/mutElk-1, sequence 5′-ctcgagcacc gggggtcctg aggattttga aggggcttaa ggctgcggtg ctgaggaagc-3′ as SEQ ID No. 36 containing a putative binding site for mut MZF-1/mut Elk-1). pGL3-60 bp PKC.alpha.-promoters, encoding the firefly luciferase (Luc) cDNA, were cotransfected with the Elk-1 or MZF-1 expression vector, alone or in combination with the truncated-Elk-1 or truncated-MZF-1 expression vector. To maintain the same DNA input in all transfection mixtures, the samples were adjusted with an empty vector (pcDNA3 or pGL3). In addition, to assess transfection efficiency, each of the DNA mixtures contained a β-galactosidase expression vector (pCH110). After incubation with DNA for 48 h, cells were washed and lysised for the measurement of luciferase activity and β-galactosidase acitivty. Luciferase activity in cell lysates was measured using Dual Luciferase Assay System (Promega) following the manufacturer's instructions. The values of Luc obtained were normalized to that of β-galactosidase to generate relative Luc units representing Elk-1 and MZF-1-dependent transcription.

### Example 12

### Electrophoretic Mobility Shift Assay is Used for Determining the Protein (MZF-1, ELK-1) and DNA (PKC-.Alpha. Promoter) Association In Vitro

The cells grown to 80% confluency were used for preparing nuclear extract by a modification of the method of Torgeman et al. (Torgeman et al., 2001, Virology, 281, 10-20). The cells were released from a 60-mm dish with trypsin/EDTA, washed with PBS, and allowed to swell on ice for 10 minutes in a buffer containing 10 mM Hepes-KOH (pH 7.9), 1.5 mM MgCl2, 10 mM KCl, 0.5 mM dithiothreitol, 0.2 mM phenylmethylsulfonyl fluoride (PMSF), and 10 mg/ml leupeptin. Nonidet P40 was then added to a 0.5% final concentration. The suspension was vigorously vortexed for 20 seconds and briefly centrifuged at 14,000 g and 4.degree C. for 15 minutes. The pellet was resuspended in a buffer containing 20 mM Hepes-KOH (pH 7.9), 25% glycerol, 420 mM NaCl, 1.5 mM MgCl₂, 0.2 mM EDTA, 0.5 mM dithiothreitol, 0.2 mM PMSF, and 10 mg/ml leupeptin. After vigorously vortexing, the suspension was placed on ice for 15 minutes before centrifuging 15,000 g and 4.degree C. for 15 minutes. The supernatants (nuclear extracts) were stored in aliquots at minus 80.degree. C. Protein concentration of the supernatants was determined by the colorimetric assay. Nuclear extracts and electrophoretic mobility shift assay (EMSA) were performed using the LightShift Chemiluminescent EMSA kit (Pierce, Rockford, Ill.). The binding reactions contained 10 μg nuclear extract protein, 10× binding buffer (10 mM Tris (pH 7.5), 50 mM KCl, 5 mM MgCl₂, 1 mM dithiothreitol, 0.05% Nonidet P-40, and 2.5% glycerol), 1 μg poly (dl-dC), and 2 (M of DNA probe). Biotin-labeled DNA duplex of sequence 5′-CCTGAGGATGGGGAAGGGGCTTCCTGCTGCGGTG-3′ as SEQ ID No. 21 containing a putative binding site for MZF-1/Elk-1 and 3′-CCTGAGGATTTTTAAGGGGCTTCCTGCTGCGGTG-5′ as SEQ ID No. 22 containing a putative binding site for mut MZF-1/Elk-1 and 5′-CCTGAGGATGGGGAAGGGGATTGGTGATGAGGTG-3′ as SEQ ID No. 23 containing a putative binding site for MZF-1/mut Elk-1 and 3′-CCTGAGGATTTTTAAGGGGATTAATGATGAGGTG-5′ as SEQ ID No. 24 containing a putative binding site for mut MZF-1/mut Elk-1 were used. The mixture was incubated at 25 degree C. for 20 minutes. After reacting, the DNA-protein complexes were subjected to a 6% native polyacrylamide gel in a 0.5× Tris borate-EDTA buffer at 100 V for 3 hours and then transferred onto a positively charged nylon membrane (Hybond-N+) in 0.5× Tris borate-EDTA buffer at 100 V for 1 hour. The membrane was immediately cross-linked for 15 minutes on a UV transilluminator equipped with 312 nm bulbs and then was detected by chemiluminescence according to the manufacturer's instructions. Supershift EMSA was also performed. The nuclear extracts obtained from SK-Hep-1 cells were incubated with the indicated biotin-labeled probes as described above.

### Example 13

### Chromatin Immunoprecipitation Assays is Used for Determining the Protein (MZF-1, ELK-1) and DNA (PKC-.Alpha. Promoter) Association In Vivo

Chromatin immunoprecipitation was performed as described (Inman et al., 2005, Mol. Cell. Biol. 25, 3182-3193) with some modifications. The SK-Hep-1 cells were harvested and cross-linked with 1% formaldehyde for 10 minutes and the reaction was terminated by the addition of 0.25M glycine. Cells were washed four times in ice-cold PBS, resuspended in RIPA lysis buffer (0.1% SDS, 1% sodium deoxycholate, 150 mM NaCl, 10 mM NaPO₄ (pH 7.2), 2 mM EDTA, 0.2 mM NaVO₃, and 1% NP-40) in the presence of Complete Protease Inhibitors (Roche Diagnostics, Mannheim, Germany), and sonicated to shear chromatin using a Cole Palmer Ultrasonic processor (Cole Palmer, Vernon Hills, Ill.). The sonicated DNA fragments were in the range of 100-1000 bp. The samples were pre-cleared with 60 μl protein A-Agarose (Sigma Chemicals, St. Louis, Mo.) for 30 minutes at 4.degree. Complexes were immunoprecipitated with 2 μg anti-Elk-1 antibody (Santa Cruz Biotechnology) or anti-MZF-1 antibody (ABGENT). The section of 210 bp PKC-.alpha. promoter containing the predicted Elk-1 and MZF-1 binding site was detected by PCR with the primers 5′-GGTACAGGCAGCTAAAACAC-3′ as SEQ ID No:25 and 5′-GTCTTCCTTCTCCCACTCC-3′ as SEQ ID No:26. The reverse Re-ChIPs was performed as following described. The pellets obtained by IP of soluble chromatin with Elk-1 and MZF-1 Abs were eluted with 500 μl of Re-ChIP buffer (0.5 mM DTT, 1% Triton X-100, 2 mM EDTA, 150 mM NaCl, 20 mM Tris-HCl pH 8.1). Next, the eluate from Elk-1 IP was precipitated with MZF-1 antibody and the eluate from MZF-1 IP was precipitated with Elk-1 antibody. The presence of the PKC-.alpha. promoter sequences in the resulting Re-ChIP pellets was examined as described above for one-step ChIP.

### SEQUENCE LISTING

<110> Chung Shan Medical University
   Liu, Jer-Yuh
   Hsieh, Yi-Hsien
<120> Methods for inhibition of proliferative disease, including hepatocellular carcinoma
<130> 0339-Dr. LIU-US
<160> 36
<170> PatentIn version 3.3
<210> 1
<211> 30
<212> DNA
<213> Homo sapiens
<220>
<221> misc_feature
<222> (1) . . . (30)
<400> 1
ttataagctt atggacccat ctgtgacgct
<210> 2
<211> 29
<212> DNA
<213> Homo sapiens

What is claimed is:

1. A method for modulating expression of PKC.-alpha. in an animal suffering a proliferative disease or condition associated with altered expression of PKC-.alpha.

2. The method of claim 1, wherein the modulating expression of PKC-.alpha. comprising an effective amount of
(a) antisense MZF-1,
(b) antisense Elk-1,
(c) vector for overexpressing polypeptide of MZF-1,
(d) vector for overexpressing polypeptide of Elk-1, or
(e) transfected cell contained (a), (b), (c) or (d).

3. The method of claim 1, wherein said proliferative disease is a cancer.

4. The method of claim 3, wherein said cancer more preferably comprising an ovary cancer, a breast cancer, a prostate cancer, a liver cancer or some other cancers expressing elevated level of PKC-.alpha.

5. The method of claim 4, wherein said cancer most preferably comprising a liver cancer, including human hepatocellular carcinoma (HCC) or some other carcinoma expressing elevated level of PKC-.alpha.

6. The method of claim 1, wherein said altered expression of PKC-.alpha. comprising increased level of PKC-.alpha. in a diseased tissue than a normal tissue.

7. The method of claim 2, wherein said antisense oligonucleotide of MZF-1 comprises the sequence in SEQ ID No: 11.

8. The method of claim 2, wherein said antisense oligonucleotide of Elk-1 comprises the sequences in SEQ ID No: 9.

9. The method of claim 2, wherein said overexpressed MZF-1 comprises SEQ. ID No: 31.

10. The method of claim 2, wherein said overexpressed Elk-1 comprises SEQ ID No: 32.

11. The method of claim 2, wherein said transfected cell is human liver cancer cell.

12. The method of claim 11, wherein said human liver cancer cell is HA22T/VGH cell or SK-Hep-1 cell.

13. A method for screening an agent effective to inhibit the development of a proliferative disease with altered level of PKC-.alpha. expression comprising
(a) incubating a mixture in a cell containing a polypeptide MZF-1, a polypeptide Elk-1, a reporter gene driven by PKC-.alpha. promoter and an agent to be tested, and
(b) identifying a potential agent comprising
(i) measuring activity of the reporter gene,
(ii) comparing the activity of the reporter gene in the absence of the agent to be tested, and
(iii) identifying a potential agent by the indication of the activity of the reporter gene in the presence of the agent.

14. The method of claim 13, wherein said proliferative disease is a cancer.

15. The method of claim 14, wherein said cancer more preferably comprising an ovary cancer, a breast cancer, a prostate cancer, a liver cancer or some other cancers expressing elevated level of PKC-.alpha.

16. The method of claim 15, wherein said cancer most preferably comprising a liver cancer, including human hepatocellular carcinoma (HCC) or some other carcinoma expressing elevated level of PKC-.alpha.

17. The method of claim 13, wherein said altered expression of PKC-.alpha. comprising increased level of PKC-.alpha. in a diseased tissue than a normal tissue.

18. The method of claim 13, wherein said polypeptide MZF-1 comprising a sequence as in SEQ ID No: 31.

19. The method of claim 13, wherein said polypeptide Elk-1 comprising a sequence as in SEQ ID No: 32.

20. The method of claim 13, wherein said reporter gene comprising luciferase, green fluorescent protein, beta-galactosidase, beta-glucuronidase, beta-lacatamase, chloramphenical acetyltransferase and other commonly used reporter genes.

21. The method of claim 20, wherein said reporter gene more suitable comprising luciferase and green fluororescense protein.

22. The method of claim 21, wherein said reporter gene the most suitable comprising luciferase.

23. The method of claim 13, wherein said promoter of PKC-.alpha. comprising sequences as in SEQ ID No: 33, 34, 35, 36.

24. The method of claim 13, wherein said cell comprising HA22T/VGH cell, SK-Hep-1 cell, Huh-7 cell, Hep3B cell, and HepG2 cell.


SEQUENCE LISTING (36 sequences)

SEQ ID NO: 1 - DNA, 30 bp, Homo sapiens
  misc_feature (1)..(30)
ttataagcttatggacccatctgtgacgct

SEQ ID NO: 2 - DNA, 29 bp, Homo sapiens
  misc_feature (1)..(29)
ttatggatcctcatggcttctggggccct

SEQ ID NO: 3 - DNA, 27 bp, Homo sapiens
  misc_feature (1)..(27)
ttataagctttgtcatgaatgaatggt

SEQ ID NO: 4 - DNA, 27 bp, Homo sapiens
  misc_feature (1)..(27)
ttatctcgagctactcggcgctgtgga

SEQ ID NO: 5 - DNA, 20 bp, Homo sapiens
  misc_feature (1)..(20)
gttctcgctggtgagtttca

SEQ ID NO: 6 - DNA, 20 bp, Homo sapiens
  misc_feature (1)..(20)
ggttttaccatcggttctgg

SEQ ID NO: 7 - DNA, 20 bp, Homo sapiens
  misc_feature (1)..(20)
tgtgggacatggagctgctg

SEQ ID NO: 8 - DNA, 20 bp, Homo sapiens
  misc_feature (1)..(20)
cagcagcaccatgtcccaca

SEQ ID NO: 9 - DNA, 21 bp, Homo sapiens
  misc_feature (1)..(21)
cagcgtcacagatgggtccat

SEQ ID NO: 10 - DNA, 21 bp, Homo sapiens
  misc_feature (1)..(21)
atggacccatctgtgacgctg

SEQ ID NO: 11 - DNA, 22 bp, Homo sapiens
  misc_feature (1)..(22)
tacacaaggggaccattcattc

SEQ ID NO: 12 - DNA, 22 bp, Homo sapiens
  misc_feature (1)..(22)
gaatgaatggtccccttgtgta

SEQ ID NO: 13 - DNA, 15 bp, Homo sapiens
  misc_feature (1)..(15)
ggggtagttgtccat

SEQ ID NO: 14 - DNA, 15 bp, Homo sapiens
  misc_feature (1)..(15)
atggacaactacccc

SEQ ID NO: 15 - DNA, 20 bp, Homo sapiens
  misc_feature (1)..(20)
ggattgttcattcttgagtc

SEQ ID NO: 16 - DNA, 20 bp, Homo sapiens
  misc_feature (1)..(20)
gactcaagaatgaacaatcc

SEQ ID NO: 17 - DNA, 21 bp, Homo sapiens
  misc_feature (1)..(21)
ggatcatcttctgccattctg

SEQ ID NO: 18 - DNA, 21 bp, Homo sapiens
  misc_feature (1)..(21)
cagaatggcagaagatgatcc

SEQ ID NO: 19 - DNA, 19 bp, Homo sapiens
  misc_feature (1)..(19)
gtcaatttccaaagcattt

SEQ ID NO: 20 - DNA, 19 bp, Homo sapiens
  misc_feature (1)..(19)
aaatgctttggaaattgac

SEQ ID NO: 21 - DNA, 34 bp, Homo sapiens
  (1)..(34): PKC alpha promoter MZF-1/Elk-1
cctgaggatggggaaggggcttcctgctgcggtg

SEQ ID NO: 22 - DNA, 34 bp, Homo sapiens
  (1)..(34): PKC alpha promoter mut MZF-1/Elk-1
cctgaggatttttaaggggcttcctgctgcggtg

SEQ ID NO: 23 - DNA, 34 bp, Homo sapiens
  (1)..(34): PKC alpha promoter MZF-1/mut Elk-1
cctgaggatggggaaggggattggtgatgaggtg

SEQ ID NO: 24 - DNA, 34 bp, Homo sapiens
  (1)..(34): PKC alpha promoter mut MZF-1/mut Elk-1
cctgaggatttttaaggggattaatgatgaggtg

SEQ ID NO: 25 - DNA, 20 bp, Homo sapiens
  misc_feature (1)..(20)
ggtacaggcagctaaaacac

SEQ ID NO: 26 - DNA, 19 bp, Homo sapiens
  misc_feature (1)..(19)
gtcttccttctcccactcc

SEQ ID NO: 27 - DNA, 25 bp, Homo sapiens
  misc_feature (1)..(25)
tgaattcatgaatggtccccttgtg

SEQ ID NO: 28 - DNA, 25 bp, Homo sapiens
  misc_feature (1)..(25)
ttctagagcagggatcctcgtccgt

SEQ ID NO: 29 - DNA, 20 bp, Homo sapiens
  misc_feature (1)..(20)
cagcagcaccatgtcccaca

SEQ ID NO: 30 - DNA, 20 bp, Homo sapiens
  misc_feature (1)..(20)
tgtgggacatggagctgctg

SEQ ID NO: 31 - protein, 72 aa, Homo sapiens
  PEPTIDE (1)..(72)
  PEPTIDE (1)..(72): flag-MZF-1 (1-72)
MNGPLVYAGFALQLGSISAGPGSVSPHLHVPWDLGMAGLSGQIQSPSREGGFAHRVLLPSDLRSEQDPTDED

SEQ ID NO: 32 - protein, 233 aa, Homo sapiens
  PEPTIDE (1)..(233)
  PEPTIDE (1)..(233): c-myc-Elk-1 (86-318)
VSYPEVAGCSTEDCPPQPEVSVTSTMPNVAPAAIHAAPGDTVSGKPGTPKGAGMAGPGGLARSSRNEYMRSGLYSTFTIQSLQPQPPPHPRPAVVLPNAAPAGAAAPPSGSRSTSPSPLEACLEAEEAGLPLQVILTPPEAPNLKSEELNVEPGLGRALPPEVKVEGPKEELEVAGERGFVPETTKAEPEVPPQEGVPARLPAVVMDTAGQAGGHAASSPEISQPQKGRKPRD

SEQ ID NO: 33 - DNA, 62 bp, Homo sapiens
  promoter (1)..(62): PKC-.alpha. -60bp
ctcgagcaccgggggtcctgaggatggggaaggggcttcctgctgcggtgctgaggaagctt

SEQ ID NO: 34 - DNA, 62 bp, Homo sapiens
  promoter (1)..(62)
  promoter (1)..(62): PKC-.alpha. -60bp mutMZF-1/Elk-1
ctcgagcaccgggggtcctgaggattttgaaggggcttcctgctgcggtgctgaggaagctt

SEQ ID NO: 35 - DNA, 62 bp, Homo sapiens
  promoter (1)..(62)
  promoter (1)..(62): PKC-.alpha. -60bp MZF-1/mutElk-1
ctcgagcaccgggggtcctgaggatggggaaggggcttaaggctgcggtgctgaggaagctt

SEQ ID NO: 36 - DNA, 62 bp, Homo sapiens
  promoter (1)..(62)
  promoter (1)..(62): PKC-.alpha. -60bp mutMZF-1/mutElk-1
ctcgagcaccgggggtcctgaggattttgaaggggcttaaggctgcggtgctgaggaagctt